(19) United States
(12) Patent Application Publication
(10) Pub. No.: US 2024/0298879 A1
(43) Pub. Date: Sep. 12, 2024

(54) ENDOSCOPE WITH SPECTRAL WAVELENGTH SEPARATOR

(71) Applicant: Lazzaro Medical, Inc., Boulder, CO (US)

(72) Inventor: Ryan Redford, Irvine, CA (US)

(21) Appl. No.: 18/601,705

(22) Filed: Mar. 11, 2024

Related U.S. Application Data
(60) Provisional application No. 63/451,476, filed on Mar. 10, 2023.

(51) Int. Cl. A61B 1/00; A61B 1/04; A61B 1/05; A61B 1/06

(52) U.S. Cl. CPC A61B 1/00096; A61B 1/000094; A61B 1/000096; A61B 1/046; A61B 1/05; A61B 1/0676

(57) ABSTRACT

An endoscope has a light source, a first lumen configured for delivering illumination from the light source to a target area, and a second lumen including a lens and a fiber-optic imaging system configured for providing real-time, images from the target area; a sleeve attached to an end of the endoscope. A beam splitter is positioned within the sleeve in front of the lens, wherein the beam splitter is configured to separate incoming light into two or more distinct wave length ranges whereby to separate imaging or analysis of different regions in the electromagnetic spectrum to separate incoming light into the visible light spectrum and the near infrared/shortwave infrared (NIR/SWIR) wavelengths. The endoscope also includes a processor configured to process and analyze light data captured by the endoscope and provide a composite image of the target area.

### CROSS REFERENCE TO RELATED APPLICATION

This application claims priority from U.S. Provisional Application Ser. No. 63/451,476, filed Mar. 10, 2023, the contents of which are incorporated herein in their entirety.

### BACKGROUND AND SUMMARY

Endoscopes are commonly used in medical and industrial settings for the inspection of internal structures and spaces by way of openings. A typical endoscope applies several modern technologies including optics, ergonomics, precision mechanics, electronics, and software engineering. By way of example, in the case of medical applications, using an endoscope, it is possible to observe lesions that cannot be detected by X-ray, making it useful in medical diagnosis. Endoscopes use tubes which are only a few millimeters thick to transfer illumination in one direction and high-resolution images in real time in the other direction, resulting in minimally invasive surgeries. It is used to examine the internal organs like the throat or esophagus. Specialized instruments are named after their target organ. Examples include the cystoscope (bladder), nephroscope (kidney), bronchoscope (bronchus), arthroscope (joints) and colonoscope (colon), and laparoscope (abdomen or pelvis). They can be used to examine visually and diagnose, or assist in surgery such as an arthroscopy. However, traditional endoscopes only capture images in the visible spectrum, which can be limited to certain applications.

The present disclosure provides an endoscope having a light source, a first lumen configured for delivering illumination from the light source to a target area, and a second lumen including a lens and a fiber-optic imaging system configured for providing real-time images from the target area with a sleeve that houses a beam splitter, which separates incoming light into different wavelength ranges. The sleeve attaches to the end of an endoscope, and the beam splitter is positioned in front of the endoscope's lens.

The beam splitter separates incoming light into two or more distinct wavelength ranges, allowing for separate imaging or analysis of different regions of the electromagnetic spectrum. For example, the beam splitter may separate light into the visible spectrum and the near NIR/SWIR wavelengths. This allows the endoscope to capture images or data in both the visible and NIR/SWIR ranges, providing additional information and capabilities.

The device can be used in a variety of medical and industrial applications, such as in minimally invasive surgery, industrial inspection, and non-destructive testing.

In accordance with one embodiment we provide an endoscope that utilizes a beam splitter to separate incoming light into two or more distinct wavelength ranges, comprising: a sleeve that attaches to the end of an endoscope, a beam splitter positioned within the sleeve in front of the endoscope's lens, wherein the beam splitter is configured to separate incoming light into two or more distinct wavelength ranges, allowing for separate imaging or analysis of different regions of the electromagnetic spectrum. The device can be used in a variety of medical and industrial applications, such as in minimally invasive surgery, industrial inspection, and non-destructive testing.

In one embodiment the beam splitter is configured to separate incoming light into the visible spectrum and the near NIR/SWIR wavelengths.

We also provide a method of using an endoscope device that utilizes a beam splitter configured to separate incoming light into two or more distinct wavelength ranges, comprising the steps of: attaching a sleeve to the end of an endoscope, positioning a beam splitter within the sleeve in front of the endoscope's lens, and capturing images or data in both the visible and the Near-Infrared (700 nm to 1400 nm) and Short-Wave Infrared (0.9-1.7 μm)-NIR/SWIR wavelength ranges by using the beam splitter to separate incoming light into two or more distinct wavelength ranges.

According to one aspect of the disclosure the beam splitter is a dichroic mirror or a prism.

In another aspect of the disclosure the device is used in medical applications such as endoscopic surgery, gastrointestinal examination, and bronchoscopy, or in industrial applications such as inspection of pipelines, engines, and other mechanical structures, or in non-destructive testing of materials, such as identifying defects and cracks in metal structures.

In one aspect of our disclosure the device is controlled by a computer, which can process and analyze the data captured by the endoscope and provide real-time feedback to the user.

In another aspect of our disclosure the device is equipped with a Near-Infrared (NIR) or Short-Wave Infrared (SWIR) imaging sensor or camera, configured to capture images or data in the NIR/SWIR wavelength range, or the device is equipped with a visible light imaging sensor or camera, configured to capture images or data in the visible wavelength range.

In a further aspect of the disclosure the device is equipped with a light source, which can be used to illuminate the object being inspected in both the visible and NIR/SWIR wavelength ranges.

In another aspect of the disclosure, we employ a hyperspectral fusion AI system configured to combine data captured in different wavelength ranges by the beam splitter and to create a composite image that provides enhanced information and capabilities.

In yet another aspect of the disclosure we employ hyperspectral fusion AI techniques to combine data captured in different wavelength ranges by the beam splitter and create a composite image.

The present disclosure also provides an endoscope device as above described, and a computer system that includes software for processing and analyzing the data captured by the endoscope, and which includes a hyperspectral fusion AI algorithm that combines data captured in different wavelength ranges by the beam splitter and creates a composite image.

In one aspect of the disclosure the computer system also includes a user interface configured to allow users to view and analyze the composite image, and adjust the settings of the endoscope device, such as wavelength range and exposure time.

The present disclosure also provides an endoscope device as above described, wherein the device is equipped with a control system configured to allow for automatic adjustment of the beam splitter settings based on the type of application and the object being inspected, with the aim of providing the best quality of image in different circumstances.

In one aspect of the disclosure the endoscope is designed to be sterilizable and reusable or is designed to be disposable and single use.

In a further aspect of the disclosure the AI system is configured to detect abnormality or disease or defects.

In a further aspect of the disclosure, we provide an endoscope device as above described and including an AI-based abnormality, disease or defect detection system that analyzes the images captured by the endoscope and identifies any potential issues.

In a still further aspect of the disclosure, we provide a method of using an endoscope device as above described and comprising the step of using AI-based abnormality, disease, or defect detection system to analyze the images captured by the endoscope and identify any potential issues.

In one embodiment the computer system also includes a user interface that allows users to view the analyzed images and receive alerts for potential issues identified by the AI-based abnormality, disease, or defect detection system.

The present disclosure also provides a system comprising an endoscope device as above described, and a computer system that includes software for processing and analyzing the data captured by the endoscope, and which includes an AI-based abnormality, disease or defect detection algorithm that analyzes the images captured by the endoscope and identifies any potential issues.

In another embodiment the endoscope device as above described includes an AI-based abnormality, disease or defect detection system which is trained on a large dataset of images, and which is continuously updated with new data to improve its accuracy and capabilities.

The present disclosure also provides an endoscope device as above described, wherein the AI-based abnormality, disease or defect detection system is also able to provide a probability score indicating the likelihood of an abnormality, disease or defect being present in the image, and/or wherein the AI-based abnormality, disease or defect detection system is also able to provide a diagnosis or recommendations for further analysis or treatment, and is trained on a large data set of images.

The present disclosure also provides an endoscope device as above described, and further comprising a topographical image interpolation technique that allows for real-time surgeon awareness.

The present disclosure also provides a method of using an endoscope device as above described, comprising the step of using topographical image interpolation techniques to generate a 3D representation of the internal structures being inspected, providing real-time awareness to the surgeon.

The present disclosure also provides a system comprising an endoscope device as above described, and a computer system that includes software for processing and analyzing the data captured by the endoscope, and which includes a topographical image interpolation algorithm that generates a 3D representation of the internal structures being inspected.

The present disclosure also provides a system as above described, wherein the computer system also includes a user interface configured to allow users to view the 3D representation of the internal structures and navigate through different layers of the image, providing a more comprehensive understanding of the internal anatomy.

The present disclosure also provides an endoscope device as above described, wherein the topographical image interpolation technique is used in combination with the hyperspectral fusion AI and the AI-based abnormality, disease, or defect detection system, whereby to provide a more comprehensive understanding of the internal structures being inspected.

In another aspect we provide an endoscope device as above described, wherein the topographical image interpolation technique is based on mathematical models such as triangulation, surface reconstruction or volumetric representation, which is described mathematically using techniques from geometry and computer graphics.

In yet another aspect of the disclosure, we provide an endoscope device as above described, wherein the topographical image interpolation technique is based on machine learning algorithms such as deep neural networks (DNN) or convolutional neural networks (CNN) that are mathematically described and analyzed using optimization and gradient descent algorithms.

We also provide an endoscope device as above described, wherein the topographical image interpolation technique is used to provide real-time feedback to the surgeon, allowing for more accurate and efficient surgical procedures.

### BRIEF DESCRIPTION OF THE DRAWINGS

Further features and advantages of the disclosure will be seen from the following detailed description, taken in conjunction with the accompanying drawings, wherein like numerals depict like parts, and wherein:

FIG. 1 is a schematic drawing of an endoscope in accordance with the present disclosure; and

FIG. 2 is a flow diagram depicting operation of the endoscope of FIG. 1 of the present disclosure.

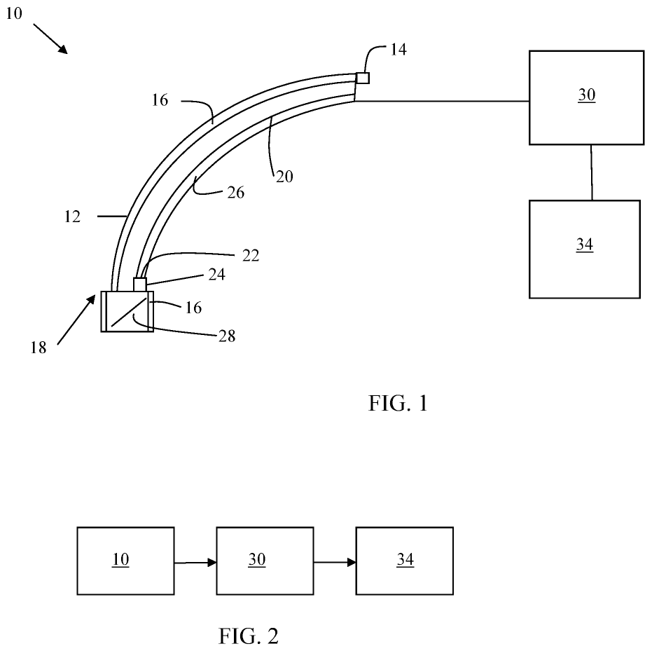

### DETAILED DESCRIPTION

An endoscope 10 in accordance with the present disclosure includes an elongate tube 12 having a light source 14 at a proximal end thereof. A first lumen 16 is configured for delivering illumination from the light source 14 to the distal end 18 of the endoscope and onto a target area. A second lumen 20 includes a lens 22 and a fiber-optic imaging system 24 configured for providing real-time images from the target area. The endoscope also includes sleeve 26 positioned over the distal end of the endoscope. A beam splitter 28 is positioned within sleeve 26 in front of lens 22 and is configured to separate incoming light from the reflective target area into two or more distinctive wavelength ranges including visible light spectrum and near infrared or short-wave infrared wavelength ranges. Completing the endoscope is a processor 30 configured to process and analyze data, i.e., light data captured by the endoscope and to provide a composite image of the target area on display 34. The endoscope also includes imaging sensors or cameras (not shown) configured to capture images or data.

Referring, in particular to FIG. 2, the images or data from the endoscope 10 are delivered to a processor 40 which is configured to employ AI to combine data captured at different wavelengths by the beam splitter and create a composite image.

The endoscope may be used wherever there is a need to capture images within an internal structure or space. These include medical uses for providing images of a patient's airway or other internal areas. The endoscope also advantageously may be used for capturing images in connection with laparoscopic procedures. Additionally, the endoscope may be used for non-medical purposes for capturing images, for example, for industrial applications.

The use of a beam splitter in an endoscope is supported mathematically through the principles of geometrical optics and wave optics. A beam splitter is modeled as a thin, partially reflective, and partially transmissive surface, which is described mathematically using Fresnel equations or the Jones matrix formalism. These mathematical models are used to predict the behavior of the beam splitter in different wavelength ranges and to optimize its design for specific applications.

The use of hyperspectral fusion AI techniques is supported mathematically through the principles of signal processing and machine learning. These techniques typically involve the use of algorithms such as principal component analysis (PCA), independent component analysis (ICA) or sparse representation (SR) to extract features from the data captured in different wavelength ranges and to create a composite image that provides enhanced information. These algorithms are mathematically described and analyzed using linear algebra, optimization, and statistical methods.

The use of AI-based abnormality, disease or defect detection is supported mathematically through the principles of computer vision, machine learning and deep learning. These techniques typically involve the use of algorithms such as convolutional neural networks (CNN) or deep neural networks (DNN) that learn to classify images into different categories (e.g. normal vs. abnormal) based on large datasets of labeled images. These algorithms are mathematically described and analyzed using optimization, gradient descent, and back-propagation algorithms.

In summary, the concepts mentioned above are mathematically supported through the principles of optics, signal processing, computer vision, machine learning, and deep learning.

One technique for fusion of data captured in different wavelength ranges is principal component analysis (PCA). PCA is a linear dimensionality reduction technique that is used to extract the most important features from a dataset of images. The PCA algorithm transforms the data into a new coordinate system, where the first principal component corresponds to the direction of maximum variance in the data. The transformed data is represented as a linear combination of the principal components, and the coefficients of this linear combination are used as a feature vector for each image.

Another technique that is used for fusion of data captured in different wavelength ranges is independent component analysis (ICA). ICA is a technique that is used to extract independent sources of information from a dataset of images. The ICA algorithm aims to find a linear transformation of the data that maximizes the non-Gaussianity of the transformed data. The transformed data is represented as a linear combination of independent components, and the coefficients of this linear combination are used as a feature vector for each image.

A third technique that is used for fusion of data captured in different wavelength ranges is sparse representation (SR). SR is a technique that is used to represent an image as a sparse linear combination of a set of basic functions. The SR algorithm aims to find the sparse representation of an image that has the smallest number of non-zero coefficients. The sparse representation is used as a feature vector for each image.

One example of an AI-based hyperspectral fusion mathematical process is the use of a convolutional neural network (CNN) for image classification. In this process, the CNN is trained on a large dataset of images captured in different wavelength ranges, such as visible, near-infrared (NIR) and short-wave infrared (SWIR). The CNN learns to extract features from the images and to classify them into different categories, such as normal vs abnormal.

The CNN is mathematically represented as a series of layers of artificial neurons, where each layer applies a set of convolutional and pooling operations to the input image. The convolutional operation is a mathematical operation that applies a set of filters to the input image, which is represented as a matrix of pixel values. The filters are also matrices that are learned during the training process. The output of the convolutional operation is a set of feature maps, which represent different aspects of the input image.

The pooling operation is a mathematical operation that reduces the size of the feature maps by taking the maximum or average value of a group of pixels. The pooling operation reduces the computational complexity of CNN and helps to make it more robust to small changes in the input image.

The output of the final layer of the CNN is a set of probabilities that indicate the likelihood of the input image belonging to different categories. The CNN is trained using a large dataset of labeled images, and the parameters of the filters and layers are learned using an optimization algorithm such as stochastic gradient descent (SGD).

One common technique for topographical image interpolation is triangulation. Triangulation is a mathematical technique that is used to represent a surface as a set of triangles. The vertices of the triangles are defined by the points on the surface, and the edges of the triangles are defined by the lines connecting the points. Triangulation is mathematically represented as a system of linear equations that describe the position of the vertices and the edges of the triangles. The system of linear equations is solved using numerical methods such as the least squares method.

Another technique that is used for topographical image interpolation is surface reconstruction. Surface reconstruction is a mathematical technique that is used to represent a surface as a set of polynomials. The polynomials describe the shape of the surface and are represented mathematically using techniques from calculus and algebra. The polynomials are fit to the data using optimization algorithms such as gradient descent or the Levenberg-Marquardt algorithm.

A third technique that is used for topographical image interpolation is volumetric representation. Volumetric representation is a mathematical technique that is used to represent a 3D object as a set of voxels (3D pixels). Voxels are represented mathematically using techniques from calculus and algebra. The voxels are fit to the data using optimization algorithms such as gradient descent or the Levenberg-Marquardt algorithm.

In regard to viewing the trachea, there are various mathematical techniques that are used to enhance the image quality and provide a more accurate view of the internal structures. Some examples include:
1. Image registration: This is a mathematical technique that is used to align multiple images captured at different angles or in different wavelength ranges. Image registration is mathematically represented as an optimization problem, where the goal is to minimize the difference between the images being aligned. Some common mathematical models used for image registration include mutual information, normalized cross-correlation, and the sum of squared differences.
2. Denoising: This is a mathematical technique that is used to reduce noise in the images captured by the endoscope. Denoising can be mathematically represented as an optimization problem, where the goal is to minimize the difference between the original image and a denoised version of the image. Some common mathematical models used for denoising include wavelet shrinkage, median filtering, and total variation denoising.
3. Segmentation: This is a mathematical technique that is used to identify and separate different structures in the images captured by the endoscope. Segmentation is mathematically represented as an optimization problem, where the goal is to minimize the difference between the image and a segmented version of the image. Some common mathematical models used for segmentation include active contours, level sets, and graph cuts.
4. Visualization: This is a mathematical technique that is used to represent the images captured by the endoscope in a 3D format. Visualization is mathematically represented as a mapping function that maps the 2D images to a 3D volume. Some common mathematical models used for visualization include volume rendering, surface rendering, and volume slicing.

In regard to tracheobronchial malacia, which is a medical condition characterized by collapse or deformity of the trachea and bronchi, there are various metaethical claims that are made regarding the use of an endoscope device as above described. These include:
1. The use of an endoscope device with a beam splitter and hyperspectral fusion AI techniques to provide a more accurate and detailed view of the trachea and bronchi, allowing for early diagnosis and treatment of tracheobronchial malacia.
2. The use of an endoscope device with a topographical image interpolation technique to provide a 3D representation of the trachea and bronchi, allowing for a more comprehensive understanding of the internal anatomy and the extent of the deformity.
3. The use of an endoscope device with an AI-based abnormality, disease, or defect detection system to provide automated and objective analysis of the images captured by the endoscope, reducing the risk of human error and improving the accuracy of the diagnosis.
4. The use of an endoscope device with real-time feedback and navigation capabilities to provide a more efficient and effective surgical procedure for the treatment of tracheobronchial malacia.
5. The use of an endoscope device that can be sterilizable and reusable or disposable and single-use to provide a more cost-effective and convenient option for the diagnosis and treatment of tracheobronchial malacia.

### Mathematical Support:

One example of an AI fusion equation is the weighted sum fusion method, which can be represented as follows:

$$Fused image = w 1 image 1 + w 2 image 2 + … + wn * image n$$

Where Fused image is the final image obtained after fusion, image1, image2, . . . , imagen are the images obtained from the different wavelength ranges, and w1, w2, . . . , wn are the weighting factors assigned to each image. The weighting factors are determined by an AI algorithm and are used to balance the contributions of each image to the final fused image.

Another example of AI fusion equation is the Dempster-Shafer fusion rule, which is a mathematical framework for combining uncertain information from different sources.

$$Belief ( A ) = Σ [ Belief ( A \vert B ) * Belief ( B ) ] for all B$$

Where Belief(A) is the belief of a given hypothesis A, Belief(A|B) is the belief of hypothesis A given hypothesis B, and Belief(B) is the belief of hypothesis B.

Summarizing to this point, the present disclosure utilizes a combination of mathematical concepts such as wavelength separation, geometric optics, and AI fusion techniques to combine the information obtained from different wavelength ranges. These equations and algorithms work together to produce a high-quality and informative final fused image.

In addition to the previous examples, other AI fusion techniques that are used in the present invention include:

### Principal Component Analysis (PCA) Based Fusion:

This method uses PCA to transform the images obtained from different wavelength ranges into a new set of uncorrelated features, and then combines the features to create a fused image.

PCA is a linear dimensionality reduction technique that can be used to transform a high-dimensional dataset into a lower-dimensional representation while preserving as much of the variance in the original data as possible. The technique is based on the eigen decomposition of the covariance matrix of the data, which results in a set of orthogonal eigenvectors (principal components) that can be used to transform the data into a new coordinate system.

The mathematical equations used in PCA is summarized as follows:
1. Compute the mean vector of the data:

$$\$ \backslash mu = \backslash frac \{ 1 \} \{ N \} \backslash sum\_ \{ i = 1 \} \hat{} \{ N \} x\_i\$$$

where $x_i$ is the $i\hat{ }{th}$ sample in the dataset, and $N$ is the total number of samples.
2. Compute the covariance matrix of the data:

$$\$ C = \backslash frac \{ 1 \} \{ N \} \backslash sum\_ \{ i = 1 \} \hat{} \{ N \} ( x\_i - \backslash mu ) ( x\_i - \backslash mu ) \hat{} T \$$$

3. Compute the eigenvectors and eigenvalues of the covariance matrix:

$$\$ Cv\_i = \backslash lambda\_i v\_i \$$$

where $v_i$ is the $i\hat{ }{th}$ eigenvector, and $\lambda_i$ is the corresponding eigenvalue.
4. Select the top k eigenvectors with the largest eigenvalues, and use them to transform the data into a new k-dimensional space:

$Z=XV$
where $X$ is the original data, $V$ is the matrix of eigenvectors, and $Z$ is the transformed data.

For PCA based fusion, the idea is to take multiple features and combine them into a new feature set. The features could be from different modalities like image, audio, text etc. The principal component analysis is applied on the combined feature set and new features are created which are linear combination of original features.

### Neural Network Based Fusion:

This method uses a neural network to learn mapping from the images obtained from different wavelength ranges to the fused image. This can be done by training a neural network on a dataset of images from different wavelength ranges, and then using the trained network to produce the fused image.

Neural network-based fusion is a technique that uses deep learning to combine multiple modalities of data, such as images, audio, and text, into a single representation that can be used for a variety of tasks such as classification, detection, and segmentation.

One way to perform neural network-based fusion is by using a multi-stream neural network architecture. In this architecture, each modality is processed by a separate stream of layers, and the outputs of these streams are then concatenated or combined in some way before being passed through a final set of layers for the task at hand. The mathematical equations that describe this process can be summarized as follows:
1. The input to the network is a set of modalities X={X1, X2, . . . , Xn}
2. Each modality Xi is processed by a separate stream of layers:

$$\$H\_i = f\_i ( X\_i ; \backslash theta\_i ) \$$$

where $f_i$ is the function implemented by the layers in the i-th stream, and $\theta_i$ are the parameters of the layers.
3. The outputs of the streams are concatenated or combined in some way:

$$\$ Z = g ( H\_ 1 , H\_ 2 , … , H\_n ) \$$$

where g is a function that concatenates or combines the outputs of the streams.
4. The concatenated or combined output Z is passed through a final set of layers for the task at hand:

$$\$ Y = h ( Z ; \backslash theta\_f ) \$$$

where h is the function implemented by the final set of layers, and $\theta_f$ are the parameters of the final layers.

Another way to perform neural network-based fusion is by using a single stream neural network architecture which has multiple branches. Each branch of the network is responsible for processing a different modality of data, and the branches are then combined by some form of element-wise operation such as addition or concatenation.

$$\$ Y = h ( f ( X\_ 1 ; \backslash theta\_ 1 ) + f ( X\_ 2 ; \backslash theta\_ 2 ) + … . f ( X\_n ; \backslash theta\_n ) ; \backslash theta\_f ) \$$$

In this type of architecture, the feature maps from each branch are combined and passed through the final layers.

In both the architectures, the function h and g can be implemented as a neural network and the parameters are learned during the training process.

### Deep Learning Based Fusion:

This method uses deep learning techniques such as convolutional neural networks (CNN) or recurrent neural networks (RNN) to combine information from different wavelength ranges. These techniques can be used to extract high-level features from the images and then use them to produce the fused image.

Deep learning-based fusion is a technique that uses deep neural networks to learn representations of multiple modalities of data, such as images, audio, and text, and combine them into a single representation that can be used for a variety of tasks such as classification, detection, and segmentation.

One way to perform deep learning-based fusion is by using a multi-stream neural network architecture, similar to the one described in the previous. In this architecture, each modality is processed by a separate stream of layers, and the outputs of these streams are then concatenated or combined in some way before being passed through a final set of layers for the task at hand. The mathematical equations that describe this process can be summarized as follows:
1. The input to the network is a set of modalities X={X1, X2, . . . , Xn}
2. Each modality Xi is processed by a separate stream of layers:

$$\$H\_i = f\_i ( X\_i ; \backslash theta\_i ) \$$$

where $f_i$ is the function implemented by the layers in the i-th stream, and $\theta_i$ are the parameters of the layers.
3. The outputs of the streams are concatenated or combined in some way:

$$\$ Z = g ( H\_ 1 , H\_ 2 , … , H\_n ) \$$$

where g is a function that concatenates or combines the outputs of the streams.
4. The concatenated or combined output Z is passed through a final set of layers for the task at hand:

$$\$ Y = h ( Z ; \backslash theta\_f ) \$$$

where h is the function implemented by the final set of layers, and $\theta_f$ are the parameters of the final layers.

Another way to perform deep learning-based fusion is by using a single stream neural network architecture which has multiple branches, similar to the one described in the previous. Each branch of the network is responsible for processing a different modality of data, and the branches are then combined by some form of element-wise operation such as addition or concatenation.

$$\$ Y = h ( f ( X\_ 1 ; \backslash theta\_ 1 ) + f ( X\_ 2 ; \backslash theta\_ 2 ) + … . f ( X\_n ; \backslash theta\_n ) ; \backslash theta\_f ) \$$$

In this type of architecture, the feature maps from each branch are combined and passed through the final layers.

In both the architectures, the function h, g and f can be implemented as a deep neural network and the parameters are learned during the training process.

In summary, deep learning-based fusion uses neural networks to learn representations of multiple modalities of data, concatenating or combining them into a single representation and using the combined representation to solve a task using a final set of layers.

### Hybrid Fusion:

This method combines multiple fusion techniques to produce the final fused image. For example, one can use PCA to extract features, a neural network to combine these features, and then a deep learning model to extract high-level features.

Hybrid fusion is a technique that combines multiple methods of data fusion, such as feature-level fusion, decision-level fusion, and score-level fusion, to improve the performance of a given task.

1. Feature-level fusion is a technique that combines the features extracted from different modalities of data at the early stages of the pipeline:

$$\$F\_ \{ hybrid \} = g ( F\_ 1 , F\_ 2 , … , F\_n ) \$$$

where F_i is the feature extracted from the i-th modality, and g is a function that combines the features, such as concatenation or element-wise addition.

2. Decision-level fusion is a technique that combines the decisions made by different classifiers on the same set of features:

$$\$D\_ \{ hybrid \} = f ( D\_ 1 , D\_ 2 , … , D\_n ) \$$$

where D_i is the decision made by the i-th classifier and f is a function that combines the decisions, such as majority voting or weighted averaging.

3. Score-level fusion is a technique that combines the scores generated by different classifiers on the same set of features:

$$\$S\_ \{ hybrid \} = h ( S\_ 1 , S\_ 2 , … , S\_n ) \$$$

where S_i is the score generated by the i-th classifier and h is a function that combines the scores, such as weighted averaging or summing.

In summary, Hybrid fusion is a combination of different fusion techniques, such as feature-level, decision-level, and score-level fusion, to improve the performance of a given task. The functions g, f, h can be implemented as neural networks and the parameters are learned during the training process.

These AI fusion techniques can improve the quality and accuracy of the fused image by combining information from different wavelength ranges in a way that is more effective than simply averaging or concatenating the images.

In summary, the present disclosure utilizes a combination of mathematical concepts, such as wavelength separation, geometric optics and AI fusion techniques, to produce a high-quality and informative fused image. These techniques include weighted sum method, Dempster-Shafer fusion rule, Principal Component Analysis, Neural Network, Deep Learning and Hybrid fusion.

What is claimed:

1. An endoscope having a light source, a first lumen configured for delivering illumination from the light source to a target area, and a second lumen including a lens and a fiber-optic imaging system configured for providing real-time, images from the target area;
a sleeve attached to an end of the endoscope;
a beam splitter positioned within the sleeve in front of the lens, wherein the beam splitter is configured to separate incoming light into two or more distinct wave length ranges whereby to separate imaging or analysis of different regions in the electromagnetic spectrum to separate incoming light into the visible light spectrum and the near infrared/shortwave infrared (NIR/SWIR) wavelengths; and
a processor configured to process and analyze light data captured by the endoscope and provide a composite image of the target area.

2. The endoscope of claim 1, wherein the beam splitter comprises a dichroic mirror or a prism.

3. The endoscope of claim 1, further comprising an NIR or SWIR imaging sensor or camera configured to capture images or data in the NIR or SWIR wavelength range.

4. The endoscope of claim 1, further comprising a visible light imaging sensor or camera configured to capture images or data in the visible wavelength range.

5. The endoscope of claim 1, wherein the processor is configured to employ a hyper-spectral fusion Artificial Intelligence (AI) system to combine data captured in different wavelength ranges by the beam splitter and to create a composite image.

6. The endoscope of claim 5, wherein the AI system is configured to combine data captured in different wavelength ranges by the beam splitter and create a composite image.

7. The endoscope of claim 1, wherein the processor includes software for processing and analyzing data captured by the endoscope and including a hyperspectral fusion AI algorithm configured to combine data captured in different wavelength ranges by the beam splitter and create a composite image.

8. The endoscope of claim 1, further comprising a user interface configured to allow users to view and analyze the composite image, and/or adjust settings of the endoscope.

9. The endoscope of claim 1, further comprising a control system configured to automatically adjust beam splitter settings based on a type of application and target being inspected.

10. The endoscope of claim 1, wherein the AI system is configured to detect abnormalities or disease or defects on the target.

11. The endoscope of claim 5, wherein the AI system is trained on a large data set of images.

12. The endoscope of claim 5, wherein the AI system is configured to provide a diagnosis or recommendation for further analyses or treatment.

13. The endoscope of claim 1, wherein the image comprises a topographical image.

14. The endoscope of claim 13, further comprising using topographical image interpretation to generate a 3D representation of the target being inspected.

15. The endoscope of claim 1, wherein the computer system includes software configured to process and analyze data captured by the endoscope and to provide a topographical image interpretation algorithm that generates a 3D representation of the target being inspected.

16. The endoscope of claim 13, further including a user interface configured to allow users to view the 3D representation.

17. The endoscope of claim 1, wherein the controller is configured to employ a mathematical model method selected from the group consisting of triangulation, surface reconstruction, and volumetric representation, to create topographical images.

18. The endoscope of claim 1, wherein the controller is configured to employ machine loading algorithms selected from the group consisting of deep neural networks and convolutional neural networks that are mathematically described and analyzed using optimization and gradient descent algorithms, to create 3D modeling images.

19. A method for inspecting internal structures comprising providing an endoscope device that utilizes a beam splitter configured to separate incoming light into two or more distinct wavelength ranges, comprising the steps of: attaching a sleeve to the end of an endoscope, positioning a beam splitter within the sleeve in front of the endoscope's lens, and capturing images or data in both the visible and the Near-Infrared (700 nm to 1400 nm) and Short-Wave Infrared (0.9-1.7 μm)—NIR/SWIR wavelength ranges by using the beam splitter to separate incoming light into two or more distinct wavelength ranges.

20. The method of claim 19, wherein the internal structure comprises an animal internal structure.
